SEQUENCE LISTING (2 sequences)

SEQ ID NO: 1 - DNA, 20 bp, Artificial Sequence
  LIPE-F
tggcacaccattttgacctg

SEQ ID NO: 2 - DNA, 21 bp, Artificial Sequence
  LIPE-R
ttgcggttagaagccacatag

(19) United States
(12) Patent Application Publication
(10) Pub. No.: US 2022/0273749 A1
(43) Pub. Date: Sep. 1, 2022

(54) PLUM EXTRACT AND METHOD FOR IMPROVING PHYSICAL FUNCTION WITH THE SAME

(71) Applicant: TCI CO., LTD., Taipei (TW)

(72) Inventors: YUNG-HSIANG LIN, Taipei (TW); WEI-CHUN LEE, Taipei (TW)

(21) Appl. No.: 17/667,562

(22) Filed: Feb. 9, 2022

Related U.S. Application Data
(60) Provisional application No. 63/153,955, filed on Feb. 26, 2021.

(30) Foreign Application Priority Data
Jan. 6, 2022 (TW) 111100633

(51) Int. Cl. A61K 36/736; A61P 3/04; A61P 1/10; A23L 33/105; A23L 33/00

(52) U.S. Cl. CPC A61K 36/736; A61P 3/04; A61P 1/10; A23L 33/105; A23L 33/30; A23V 2002/00

(57) ABSTRACT

Provided is a method for improving physical function, including administering to a subject in need thereof a composition including a plum extract extracted from unripe green plums that are harvested within two to three months after flowering. The plum extract is used for inhibiting accumulation of lipid droplets in cells, increasing a metabolic rate, improving gastrointestinal function, and reducing a weight and/or a waist circumference.

### CROSS-REFERENCES TO RELATED APPLICATIONS

This application claims the benefit of U.S. provisional application Ser. No. 63/153,955, filed on Feb. 26, 2021 and claims the priority of Patent Application No. 111100633 filed in Taiwan, R.O.C. on Jan. 6, 2022. The entirety of the above-mentioned patent applications are hereby incorporated by references herein and made a part of the specification.

### REFERENCE OF AN ELECTRONIC SEQUENCE LISTING

The contents of the electronic sequence listing (P211772USI_ST25.txt; Size: 658 bytes: and Date of Creation: Jan. 26, 2022) is herein incorporated by reference in its entirety.

### BACKGROUND

### Technical Field

The present invention relates to an extract, and in particular, to a method for preparing a composition for improving physical function with a plum extract from unripe green plums within two to three months after flowering.

### Related Art

A plum is a fruit of the genus Prunus family Rosaceae. Plums are usually harvested between July and August, and can be eaten freshly or canned. The plum has been listed as the first of the “five fruits” since ancient times, and it is also praised as a fruit that can beautify the skin by the Compendium of Materia Medica.

Ripe plums taste sweet and sour with soft flesh and plenty of juice, which are very popular with consumers. Plum trees are also the favorite economic crops for farmers with strong adaptability to the climate and rapid growth without rigorous conditions on soil.

### SUMMARY

However, unripe plums taste sour with firm flesh, making them inedible. Based on this, the present invention provides use of a plum extract from unripe green plums within two to three months after flowering for preparing a composition for improving physical function. The present invention provides a method for improving physical function with a plum extract, including administering to a subject in need thereof a composition including a plum extract from unripe green plums within two to three months after flowering. The unripe green plum fruits are further utilized to enhance its overall industrial value, and overcome overproduction in industry or reutilize the unripe green plum fruits removed during fruit thinning.

In some embodiments, the improving physical function involves increasing an expression level of LIPE gene, a fat loss-related gene.

In some embodiments, improving physical function involves inhibiting accumulation of lipid droplets in cells.

In some embodiments, improving physical function involves increasing a metabolic rate.

In some embodiments, increasing a metabolic rate involves increasing an efficiency of pyruvic acid production.

in some embodiments, improving physical function involves reducing a weight and/or a waist circumference.

In some embodiments, improving physical function involves improving gastrointestinal function. In some embodiments, improving gastrointestinal function involves relieving one of symptoms such as abdominal distension, nausea, and regurgitation, in some embodiments, improving gastrointestinal function involves improving one of symptoms such as intestinal peristalsis, relieving constipation, and softening feces.

In some embodiments, a composition is a food composition including at least 2 g/day of the plum extract That is, an effective dose of the plum extract is 2 g/day.

Based on the above, the plum extract according to any embodiment can improve physical function. The plum, extract according to any embodiment can increase an expression level of a fat loss-related gene, that is, the LIPE gene, to improve physical function. The plum extract according to any embodiment can inhibit accumulation of lipid droplets in cells to improve physical function. The plum extract according to any embodiment can increase a metabolic rate by increasing an efficiency of pyruvic acid production to improve physical function. The plum extract according to any embodiment can reduce weight and/or waist circumference to improve physical function. The plum extract according to any embodiment can improve gastrointestinal function to improve physical function. In addition, the plum extract according to any embodiment cart effectively improve physical function by taking 2 g of plum extract per day.

### BRIEF DESCRIPTION OF THE DRAWINGS

FIG. 1 is a graph of comparison in the content of flavonoids between a plum extract according to an embodiment of the present invention and a ripe plum extract;

FIG. 2 is a graph of comparison in the content of tannin between a plum extract according to an embodiment of the present invention and a ripe plum extract;

FIG. 3 is a graph of comparison in the content of flavonoids between a plum extract according to an embodiment of the present invention and another variety of green fruit extract;

FIG. 4 is a graph of comparison in the content of tannin between a plum extract according to an embodiment of the present invention and another variety of green fruit extract;

FIG. 5 is a graph showing test results of an expression level of the LIPE gene of a plum extract according to an embodiment of the present invention;

FIG. 6 shows the distribution status of lipid droplets for a plum extract according to an embodiment of the present invention and a ripe plum extract in the test for the accumulation of lipid droplets in fat cells;

FIG. 7 is a graph of comparison in the results of the test for the accumulation of lipid droplets in fat cells between a plum extract according to an embodiment of the present invention and a ripe plum extract;

FIG. 8 is a graph of comparison in the results of the test for the production rate of pyruvic acid between a plum extract according to an embodiment of the present invention and a ripe plum extract;

FIG. 9 is a graph showing the results of changes in weight in a human subject experiment;

FIG. 10 is a graph showing the results of changes in waist circumference in a human subject experiment;

FIG. 11 is a graph showing the results of alleviation of overall gastrointestinal discomfort by questionnaire in a human subject experiment;

FIG. 12 is a graph showing the results of alleviation of upper abdominal fullness by questionnaire in a human subject experiment;

FIG. 13 is a graph showing the results of alleviation of nausea by questionnaire in a human subject experiment;

FIG. 14 is a graph showing the results of alleviation of regurgitation by questionnaire in a human subject experiment;

FIG. 15 is a graph showing the results of the frequency of intestinal peristalsis by questionnaire in a human subject experiment;

FIG. 16 is a graph showing the results of ease of defecation by questionnaire in a human subject experiment; and

FIG. 17 is a graph showing the results of feces softening by questionnaire in a human subject experiment.

### DETAILED DESCRIPTION

The present invention provides a method for preparing a composition for improving physical function with a plum extract from unripe green plums within two to three months after flowering.

In some embodiments, a plum extract is extracted from a plum raw material with a solvent.

In some embodiments, a plum raw material refers to unripe green plums within two to three months after flowering from the Prunus sect. Prunus. In some embodiments, a plum raw material is prepared from the Prunus salicina. In some embodiments, a plum raw material refers to whole fruits including the skin, the flesh, and the pyrena. In some embodiments, a plum raw material refers to fresh plums, dried plums, frozen plums, or plums that are processed by other physical methods to facilitate handling. In some embodiments, a plum raw material may be whole plums, minced plums, diced plums, milled plums, ground plums, or plums processed by other methods for altering the size and physical integrity. For example, the plum raw material is obtained b coarsely crushing green plums by using a machine with a pore size of 30 mm.

In an embodiment, a solvent may be pure water. In some embodiments, a weight ratio of solvent to plum raw material is 5-20:1-5. In some embodiments, a weight ratio of solvent to plum raw material is 5:1.

In some embodiments, a step of extraction refers to mixing a solvent and a plum raw material, and then heating to a set temperature to be kept for a fixed period of time. In some embodiments, a set temperature may be 85±5° C. In sonic embodiments, a fixed period of time may be 60-90 min, In some other embodiments, a step of extraction refers to mixing a solvent and a plum raw material, and then heating to a set temperature to be kept for a fixed period of time. If the amount of the solvent is excessively small or the fixed period of time is excessively short, the extraction efficiency will be significantly reduced. if the extraction time is excessively long, the active ingredients in the extract may be degraded.

In some embodiments, there is a step of filtering after the step of extraction The step of filtering refers to filtering out solids from the plum raw material and solvent after the step of extraction with a filter to form a filtrate. For example, the filter may be a 400-mesh filter, in some embodiments, a step of filtering refers to first centrifuging the plum raw material and solvent after the step of extraction to obtain a supernatant, and then filtering out solids from the supernatant with a filter to form a filtrate.

In some embodiments, there is a step of cooling between the step of extraction and the step of filtering. The step of cooling refers to naturally cooling the heated plum raw material and solvent down to room temperature (25-30° C.).

In some embodiments, a plum extract is extracted from a plum raw material through extraction with a solvent, filtering, and concentration under reduced pressure.

In sonic embodiments, a step of concentration is carried out by a concentrator under reduced pressure at 45-70° C. In some embodiments, a step of concentration is carried out by a concentrator under reduced pressure at 60±5° C. For example, the concentrator may be a concentrator with a brand/model: BUCHI-Rota vapor R-100. In some embodiments, a step of concentration is carried out until the liquid is concentrated to a degrees Brix of 5.0±0.5. In some embodiments, concentration is carried out under reduced pressure set to 150 bar. Herein, concentration under reduced pressure can remove alcohol from the liquid and reduce the storage volume of the liquid.

In some embodiments, improving physical function refers to increasing an expression level of a fat loss-related gene. The fat loss-related gene refers to the hormone-sensitive lipase (LIPE, gene ID 3991) gene. The LIPE gene was transcribed to produce hormone-sensitive lipase (HSL). The HSL can hydrolyze diglycerides into monoglycerides, which plays a pivotal role in lipolysis.

In some embodiments, improving physical function involves inhibiting accumulation of lipid droplets in cells.

In some embodiments, improving Physical function involves increasing, a metabolic rate. In some embodiments, increasing a metabolic rate involves increasing an efficiency of pyruvic acid production.

In some embodiments, improving physical function involves reducing a weight and/or a waist circumference.

In some embodiments, improving physical function involves improving gastrointestinal function. In some embodiments, improving gastrointestinal function involves relieving one of symptoms such as abdominal distension, nausea, and regurgitation. In some embodiments, improving gastrointestinal function involves improving, one of symptoms such as intestinal peristalsis, relieving constipation, and softening feces.

In some embodiments, a composition is a food composition including at least 2 g/day of the plum extract. That is, an effective dose of the plum extract is 2 g/day.

In some embodiments, the foregoing composition may be a medicament. In other words, the medicament includes an effective dose of plum extract

In some embodiments, the foregoing medicament may be manufactured into a dosage form suitable for enteral, parenteral, oral, or topical administration using techniques well known to those skilled in the art.

In sonic embodiments, the dosage form for enteral or oral administration includes, but is not limited to: a tablet, a troche, a lozenge, a pill, a capsule, a dispersible powder or granule, a solution, a suspension, an emulsion, a syrup, an elixir, a slurry, or other similar substances. In some embodiments, the dosage form for parenteral or topical administration includes, but is not limited to: an injection, a sterile powder, an external preparation, or other similar substances. In some embodiments, the administration manner of the injection may be subcutaneous injection, intraepidermal injection, intradermal injection, or intralesional injection.

In some embodiments, the foregoing medicament may include a pharmaceutically acceptable carrier widely used in drug manufacturing technology. in some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, or other similar substances. The type and quantity of selected carriers fall within the scope of professionalism and routine technology of those skilled in the art. in some embodiments, the solvent of the pharmaceutically acceptable carrier may he water, normal saline, Phosphate buffered saline (PBS), or aqueous solution containing alcohol.

In some embodiments, the foregoing composition may be an edible product (that is, a food composition). In other words, the edible product includes a specific content of plum extract. In some embodiments, the edible product may be a general food, food for special health use (FoSHU), dietary supplement, or food additive.

In some embodiments, the foregoing edible product may be manufactured into a dosage form suitable for oral administration using techniques well known to those skilled in the art. In some embodiments, the general food may be, but is not limited to: beverages, fermented foods, bakery products, or condiments.

In some embodiments, the plum extract (as a food additive) of any embodiment can be added during the preparation of raw materials by conventional methods, or the plum extract (as a food additive) of any embodiment is added in the food preparation process to be prepared with any edible material into an edible product for humans and non-human animals to eat.

### Example 1: Preparation of Samples

Plum extract sample 1: Unripe green plums (Prunus salicina) were crushed to obtain a plum raw material. Herein, the green plums were whole frozen plums including the skin, the flesh, and the pyrena. Herein, the green plums were crushed by a 10-speed blender (brand: osterizer) with a. pore size set to 30 mm. Next, the crushed plum raw material was mixed with water as a solvent in a weight ratio of 1:5, then subjected to extraction at 85° C. for 1 h, and then cooled down to room temperature (25° C.). Then, the: cooled plum raw material and solvent were centrifuged to obtain a supernatant, and the supernatant was filtered with a 400-mesh filter to form a filtrate. Herein, the cooled plum raw material and solvent were centrifuged by a centrifuge with a brand/model: Thermo Scientific Heraeus Fresco 17. Then, the filtrate was concentrated under reduced pressure at 60° C. to a degrees Brix of 5.0±0.5 to obtain the plum extract sample. Herein, the. filtrate was concentrated under reduced pressure by a concentrator with a brand/model: BUCHI-Rotavapor R-100.

Plum extract sample 2: Prepared by mixing 95.79% of water, 4% of plum extract sample 1, 0.2% of citric acid, and 0.01% of sucralose.

Control sample A: Dry ripe red plums (Prunus salicia) were coarsely crushed by a blender with a pore size of 30 mm to obtain a plum raw material. Herein, the red plums were crushed by a 10-speed blender (brand: osterizer). Next, the crushed plum raw material was mixed with water as a solvent in a weight ratio of 1:5, then subjected to extraction at 85° C. for 1 h, and then cooled down to room temperature (25° C.). Then, the cooled plum raw material and solvent were centrifuged to obtain a supernatant, and the supernatant was filtered with a 400-mesh filter to form a filtrate. Herein, the cooled plum raw material and solvent were centrifuged by a centrifuge with a brand/model. Thermo Scientific. Heraeus Fresco 1.7. Then, the filtrate was concentrated under reduced pressure at 60° C. to a degrees Brix of 5.0±0.5 to obtain the control sample A. Herein, the filtrate was concentrated under reduced pressure by a concentrator with a brand/model: BUCHI-Rotavapor R-100.

Control sample B: Another variety of unripe green plums (water plums, also referred to as Australian plums and Oriental plums, Prunus saliciana) were crushed by a blender and then filtered with a 10-mesh filter to obtain a plum raw material. Herein, the red plums were crushed by a 10-speed blender (brand: osterizer). Next, the crushed plum raw material was mixed with water as a solvent in a weight ratio of 1:5, then subjected to extraction at 85° C. for 1 h, and then cooled down to room temperature (25° C). Then, the cooled plum raw material and solvent were centrifuged to obtain a supernatant, and the supernatant was filtered with a 400-mesh filter to form a filtrate. Herein, the cooled plum raw material and solvent were centrifuged by a centrifuge with a brand/model: Thermo Scientific Heraeus Fresco 17. Then, the filtrate was concentrated under reduced pressure at 60° C. to a degrees Brix of 5.0±0.5 to obtain the control sample B. Herein, the filtrate was concentrated under reduced pressure by a concentrator with a brand/model: BUCH1-Rotavapor R-100.

### Example 2: Comparison in Content between Plum Extract and Ripe Plum Extract

2.1. Test of Content of Flavonoids

The control sample A obtained in the Example 1 was taken as a control sample, and the plum extract sample 1 obtained in the Example 1 was taken as an experimental sample. Each sample was diluted 20-fold with water to 1200 μL. 200 μL of 5% sodium nitrite was mixed in to stand for 6 min, next, 200 μL. of 10% aluminum nitrate was mixed in to stand for 6 min, 2 mL of 4% sodium hydroxide was mixed in, and finally 1.4 mL of water was mixed in, to obtain a to-be-tested reaction solution. The to-be-tested reaction solution was transferred into a 96-well plate, and an absorbance at 500 nm of the to-be-tested reaction solution was measured by a spectrophotometer.

In addition, a standard curve was plotted with rutin as a reference. Herein, 0 μg/mL, 400 μg/mL, 600 μg/L, 1000 μg mL, and 1200 μg/mL reference solutions of rutin were separately prepared. For each reference solution, 200 μL of 5% sodium nitrite was mixed in to stand for 6 min, next, 200 μL, of 10% aluminum nitrate was mixed in to stand for 6 min, then 2 mL of 4% sodium hydroxide was mixed in, and finally 1.4 mL of water was mixed in, to obtain a to-be-tested reference solution. 200 μL of the to-be-tested reference solution was transferred into a 96-well plate, an absorbance at 500 nm of each to-be-tested reference solution was measured by a spectrophotometer, respectively 0.035 for 0 μg/mL, 0.13 for 400 μg/mL, 0.183 for 600 μg/mL, 0.273 for 1000 and 0.335 for 1200 μg/mL, and the results was calculated based on linear regression to obtain the standard curve.

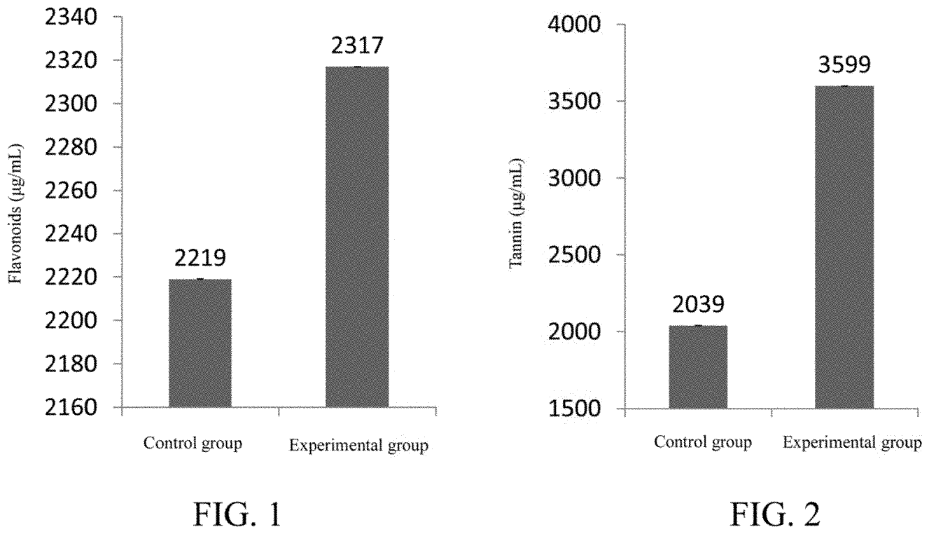

Then, the absorbance of the to-be-tested reaction solution was converted into the content of flavonoids based on the standard curve. Herein, as shown in FIG. 1, the content of flavonoids of the control sample A (control group) is 2219 μg/mL, and the content of flavonoids of the plum extract sample 1 (experimental group) is 2317 μg/mL.

As, shown in FIG. 1, the experimental results show that the content of flavonoids of the plum extract in the embodiments of the present invention is higher than the content of flavonoids of the prepared control sample A. Based on this, the composition of unripe green plums is not the same as that of ripe red plums.

2.2. Test of Content of Tannin

The control sample A obtained in the Example 1 was taken as a control sample, and the plum extract sample 1 obtained in the Example 1 was taken as an experimental sample.

1 mL of each sample was contained into a 25 mL volumetric flask, and then precisely adjusted in volume with ultrapure water. Next, 10 μL of each sample was mixed uniformly with 750 μL of ultrapure water. Then, 50 μL of Folin & Ciocalten's phenol reagent was mixed in to stand for 8 Min, and then 100 μL of saturated sodium carbonate solution was mixed in to react at room temperature for 2. h, obtain a to-be-tested sample solution. 200 μL of the to-be-tested sample solution was transferred into a 96-well plate, and an absorbance at 765 nm of the to-be-tested sample solution was measured by a spectrophotometer.

In addition, a standard curve was plotted with tannic acid as a reference. Herein, 0 ppm, 100 ppm. 200 ppm, 600 ppm, and 800 ppm reference solutions of tannic acid were separately prepared. 10 μL of each reference solution was mixed uniformly with 750 μL of ultrapure water. Then, 50 μL of Folin & Ciocalten's phenol reagent was mixed in to stand for 8 min, and then 100 μL of saturated sodium carbonate solution was mixed in to react at room temperature for 2 h, to obtain a to-be-tested reference solution. 200 μL of the to-be-tested reference solution was transferred into a 96-well plate, an absorbance at 765 nm of each to-be-tested reference solution was measured by a spectrophotometer, and the measurement results was calculated based on linear regression to obtain the standard curve.

Then, the absorbance of the to-be-tested reaction solution was converted into the content of tannic acid based on the standard curve. Herein, the content of tannin of the control sample A (control group) is 2039 μg/mL, and the content of tannin of the plum extract sample 1 (experimental group) is 3559 μg/mL.

As shown in FIG. 2, the experimental results show that the content of tannin of the plum extract in the embodiments of the present invention is higher than the content of tannin of the prepared control sample A. Based on this, the composition of unripe green plums is not the same as that of ripe red plums.

### Example 3: Comparison in Content between Plum Extract and Another Variety of Green Fruit Extract

3.1, Test of Content of Flavonoids

The control sample B obtained in the Example 1 was taken as a control sample, and the plum extract sample I obtained in the Example 1 was taken as an experimental sample.

Refer to 2.1 above for the test process with the same experimental steps and experimental equipments.

Finally, the absorbance of the to-be-tested reaction solution was converted into the content of flavonoids based on the standard curve. Herein, the content of flavonoids of the control sample B is 1007 μg/mL, and the content of flavonoids of the plum extract sample 1 is 2307 μg/mL.

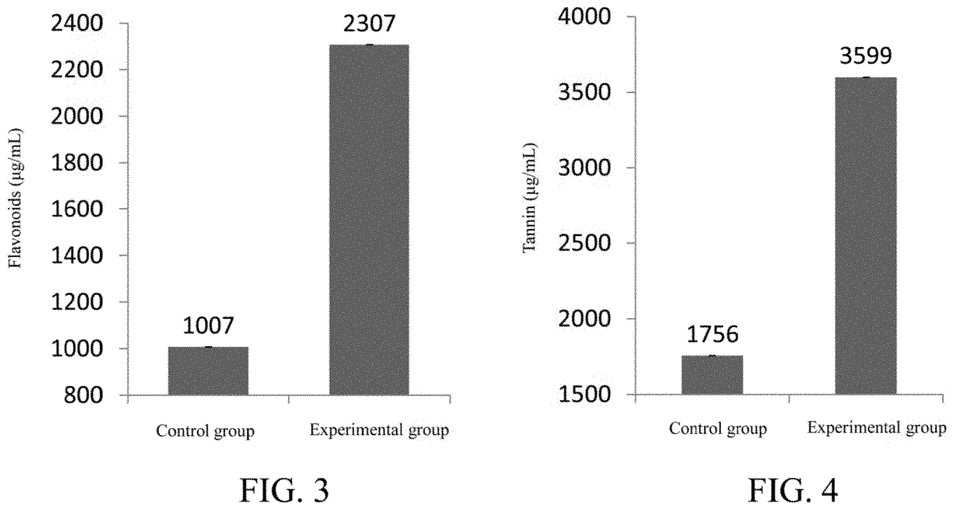

As shown in FIG. 3, the experimental results show that the content of flavonoids of the plum extract in the embodiments of the present invention is higher than the content of flavonoids of the prepared control sample B. Based on this, the composition of unripe green plums is not the same as that of another variety of unripe green plums.

3.2. Test of Content of Tannin

The control sample B obtained in the Example 1 was taken as a control sample, and the plum extract sample 1 obtained in the Example 1 was taken as an experimental sample.

Refer to 2.2 above for the test process with the same experimental steps and experimental equipments.

Finally, the absorbance of the to-be-tested reaction solution was converted into the content of tannic acid based on the standard curve. Herein, the content of tannin of the control sample B is 1756 μg/mL, and the content of tannin of the plum extract sample 1 is 3599 μg/mL.

As shown in FIG. 4, the experimental results show that the content of tannin of the plum extract in the embodiments of the present invention is higher than the content of tannin of the prepared control sample B. Based on this, the composition of unripe green plums is not the same as that of another variety of unripe green plums.

### Example 4. Test of Expression Level of Fat Loss-Related Gene 4.1, Material, instrument, and solution preparation

Experimental cell: mouse bone marrow stromal cells (hereinafter referred to as OP9 cells) of the OP9 cell strain (ATCC CRL-2749) purchased from the American Type Culture Collection (ATCC®).

Culture medium: α-minimum essential medium (α-MEM, Gibco, number 12000-022) containing 20% of fetal bovine serum (FBS, Gibco, number 10438-026, USA) and 1% of antibiotic-antimycotic (Gibco, number15240-062):

Reagent: RNA extraction reagent kit (purchased from Geneaid, Taiwan, Lot No. FC24015-G), and KAPA SYBR® FAST qPCR reagent kit (purchased from Sigma, USA, number 38220000000).

Reverse transcriptase: SuperScript® III Reverse Transcriptase Invitrogen, USA, number 18080-051).

Detection instrument AB StepOnePlus™ Real-Time PCR system (purchased from the Thermo Fisher Scientific, USA).

4.2, Test Process

First, 1.5×10 ⁵ Op9 cells were inoculated into a cell culture plate containing 2 mL of the foregoing culture medium per well, and then cultured at 37° C. for 24 h, and the cultured OP9 cells were divided into a blank group, a control group, and an experimental group for different treatment methods.

No test samples were added into the blank group, the control sample A obtained in the Example 1 was taken as a control sample, and the plum extract sample 1 obtained in the

Example 1 was taken as an experimental sample. Herein, the test samples were respectively added into the culture medium of the control group and the experimental group in a concentration of 0.025 mg/mL.

After the control group and the experimental group were cultured at 37° C. for 24 h, the cell membranes were broken with a cell lysis buffer to form a cell solution.

Next, RNA of the two groups of cell solutions was collected separately by using an RNA extraction reagent kit. Then, 1000 ng of the extracted RNA in each group was used as a template, and reverse transcription was carried out through binding with primers shown in Table 1 by using the reverse transcriptase to produce corresponding cDNA. Subsequently, the quantitative real-time reverse transcription polymerase chain reaction was carried out on the two groups of reverse transcribed products respectively with the primer combination in Table 1 by using the real-time PCR system and the qPCR reagent kit to observe the expression level of gene in the cells in each group. the instrument setting conditions for the quantitative real-time reverse transcription polymerase chain reaction were 95° C. for 1 s, 60° C. for 20 s, a total of 40 cycles, and gene quantification b the 2-ΔCt method. Therefore, the quantitative real-time reverse transcription polymerase chain reaction with cDNA can indirectly quantify the mRNA expression level of each gene, and then infer the expression level of the protein encoded by each gene.

TABLE 1
(F represents a forward primer, and R represents a reverse primer)

| | | Primer | | Primer |
|---|---|---|---|---|
| Gene | Number | name | Sequence (5′→3′) | length |
| LIPE | SEQ ID 1 | LIPE-F | TGGCACACCATTTTGACCTG | 20 |
| | SEQ ID 2 | LIPE-R | TTGCGGTTAGAAGCCACATAG | 21 |

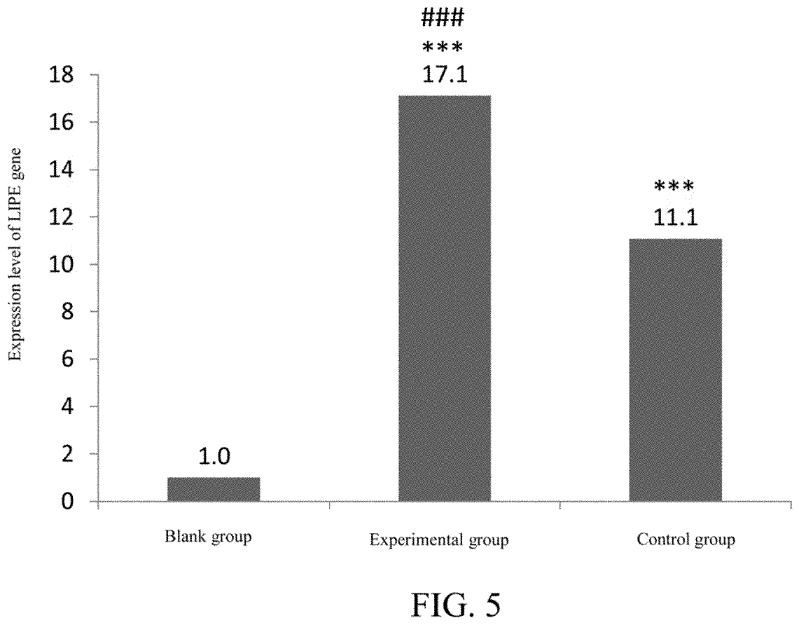

As shown in FIG. 5, the relative gene expression shown in FIG. 5 is presented in relative scale. The standard deviation is calculated by using the STDEV formula of Excel software, and whether there is a statistically significant difference is analyzed by one-tailed student t-test in Excel software.

In the figure, “*” represents a p value less than 0.05, “**” represents a p value less than 0.01, and “***” represents a p value less than 0.001. More “*” represents more significant statistical differences from the blank group.

In the figure, “#” represents a p value less than 0.05, “##” represents a p value less than 0.01, and “###” represents a p value less than 0.001. More “#” represents more significant statistical differences from the control group.

4.3. Test Results

Referring to 5, when an expression level of the LIPE gene in the blank group was regarded as 1 (that is, 100%), an expression level of the LIPE gene in the control group relative to the blank group was 11.1 (that is, 111%), and an expression level of the LIPE gene in the experimental group relative to the blank group was 17.1 (that is, 171%), indicating that the expression level of the LIPE gene in the experimental group was 17 folds of that in the blank group.

In addition, under the calculation of statistical t-test, the experimental group and the control group both have significant differences from the blank group, and the experimental group also has significant differences from the control group.

That is, both the plum extract or the ripe red plum extract have a significant effect of increasing the expression level of the LIPE gene, and the effect of the plum extract is significantly greater than that of the ripe red plum extract.

### Example 5: Test of Accumulation of Lipid Droplets in Cells

Fat is stored in fat cells in the form of lipid droplets. Based on this, stained lipid droplets were analyzed in this test to observe the quantity of the lipid droplets in the cells to further confirm the status of fat accumulation, Subsequently, the dye was dissolved out and then quantified as a numerical indicator.

5.1 Material or Solution Preparation

Experimental cell: Mouse bone marrow stromal cells (hereinafter referred to as OP9 cells) of the OP9 cell strain (ATCC CRL-2749) purchased from the American Type Culture Collection ATCC®).

Culture medium: minimum essential medium alpha medium (MEMAM, purchased from Gibco, number Cat. 12000-022) containing 20% of fetal bovine serum (FBS, purchased from Gibco, number Cat. 10437-028) and 0.1% of penicillin-streptomycin (purchased from Gibco, number Call 5240-062).

5.2. Test Process

First, 8×10⁴ OP9 cells were inoculated into a 24-well culture plate containing 500 μL, of the culture medium per well, and then cultured at 37° C. for 7 days. The culture medium was replaced every three days during the seven days of cell culture. After the seven days, the formation of lipid droplets in the cells was observed by using a microscope (ZEISS; magnification 400×) to confirm that the cells were fully differentiated into fat cells. The fat cells obtained after the differentiation were divided into five groups: a blank group, a control group 1, a control group 2, an experimental group 1, and an experimental group 2.

Experimental group 1: The plum extract sample 1 prepared by the method in the Example 1 was added into the cells obtained after the differentiation in a concentration of 0.25 mg/mL, and then cultured at 37° C. for 7 days. The culture medium and the sample were replaced every three days during the seven days of cell culture.

Experimental group 2: The plum extract sample 1 prepared by the method in the Example 1 was added into the cells obtained after the differentiation in a concentration of 0.125 mg/mL, and then cultured at 37° C. for 7 days. The culture medium and the sample were replaced every three days during the seven days of cell culture.

Control group 1: The control sample A prepared by the method in the Example 1 was added into the cells obtained after the differentiation in a concentration of 0.25 mg/mL, and then cultured at 37° C. for 7 days. The culture medium and the sample were replaced every three days during the seven days of cell culture.

Control group 2: The control sample A prepared by the method in the Example 1 was added into the cells obtained after the differentiation in a concentration of 0.125 mg/mL, and then cultured at 37° C. for 7 days. The culture medium and the sample were replaced every three days during the seven days of cell culture.

Blank group: without any treatment, that is, no additional compounds were added into the culture medium containing the fat cells obtained after the differentiation, and then cultured at 37° C. for 7 days. The culture medium was replaced every three days during the seven days of cell culture.

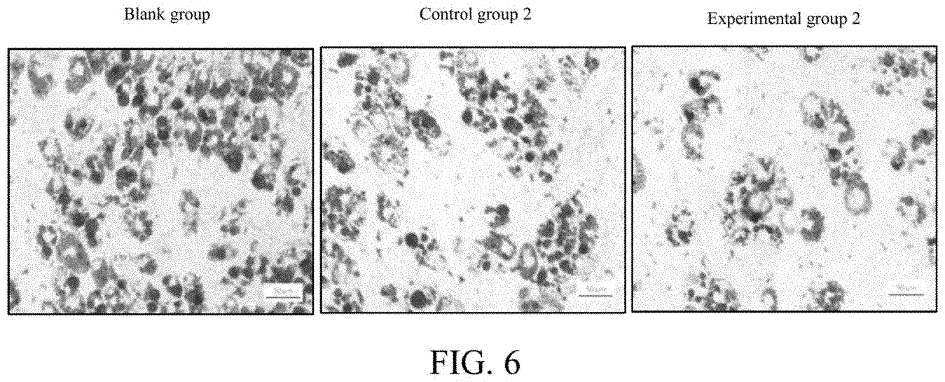

Next, staining was carried out with Oil Red O according to the following steps. After the seven days of cell culture, the culture medium was removed, the fat cells were washed with I mL of phosphate buffered saline (PBS) two times, and then 1 of 10% formaldehyde was added to react at room temperature for 30 min to immobilize the fat cells. After the formaldehyde was removed, the fat cells were slightly washed with 1 mL of PBS two times. Then, 1 of 60% isopropanol was added into the cells in each well to react for 1 min. After that, the isopropanol was removed, and 1 mL of Oil Red O solution was added to react with the fat cells at room temperature for 1 b. Then, the Oil Red 0 solution was removed, and the fat cells were then rapidly destained with 1 mL of 60% isopropanol for 5 s. The cells in the blank group, the control group 2, and the experimental group 2 were observed and photographed by using a microscope. The photographed cells are shown in FIG. 6.

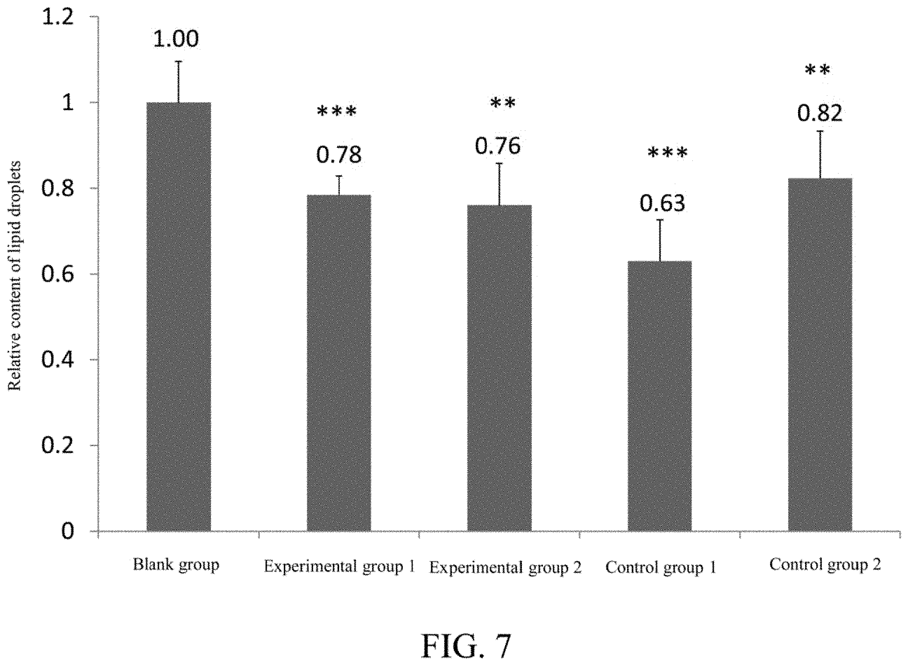

Subsequently, each stained group was quantified for Oil Red O according to the following steps. 100% isopropanol was added into the stained cells, and then placed on a shaker to react for 10 min to dissolve lipid droplets. Then, 100 μL of the reacted cell solution was transferred into a 96-well culture plate and then measured by an ELISA reader (BioTek) to obtain a value at OD510 nm of each group. As shown in FIG. 7, the obtained results were analyzed by student t-test using Excel software to determine whether there is a statistically significant difference between two sample groups. (In the figure, “*” represents a p value less than 0.05, “*” represents a p value less than 0.01, and “***” represents p value less than 0.001. More “*” represents more significant statistical differences.)

5.3. Test Results.

Referring to FIG. 6, in the control group 2, after the treatment with the ripe red plum extract, the quantity of lipid droplets is significantly less than that in the blank group; and in the experimental group 2, after the treatment with the plum extract of the present invention, the quantity of lipid droplets was significantly less than that in the blank group and the control group. In other words, the plum extract effectively reduced the accumulation of fat in mature fat cells.

Referring to FIG. 7, in a case that the accumulation of lipid droplets in the blank group was regarded as 1, the accumulation of lipid droplets in the experimental group 1 relative to the blank group was 0.78, indicating that the fat accumulation was reduced by 22% in the case of the concentration of the plum extract of 0.25 mg/mL; the accumulation of lipid droplets in the experimental group 2 relative to the blank group was only 0.76, indicating that the fat accumulation was reduced by 24% in the case of the concentration of the plum extract of 0.125 mg/mL, the accumulation of lipid droplets in the control group 1 relative to the blank group is 0.63, indicating that the fat accumulation was reduced by 37% in the case of the concentration of the ripe plum extract of 0.25 mg/mL; and the accumulation of lipid droplets in the control group 2 relative to the blank group was 0.82, indicating that the fat. accumulation was reduced by 18% in the case of the concentration of the ripe plum extract of 0.125 mg/mL. It can be learned that the plum extract effectively inhibited the accumulation of fat, and reduced fat formation of subjects, thereby achieving weight loss.

### Example 6: Test of Production Rate of Pyruvic Acid

Herein, the change of the basal metabolic rate of mouse myoblasts C2C12 treated with the plum extract was determined by the amount of pyruvic acid, the final product of metabolism.

6.1. Material and Instrument

Experimental cell: mouse myoblasts C2C12 from the Bioresource Collection and Research Center (BCRC.; Cat. 60083).

Culture medium: Dulbecco's Modified Eagle's Medium (DMEM, purchased from Gibco, 12100-046) containing 10 vol % of EBS (purchased from Gibco, 10437-028) and 1% of antibiotic (purchased from Gibco, Cat. 15240-062).

Experimental solution: PBS solution (purchased from Gibco, number 10437-028), horse serum (purchased from Gibco, number Cat. 16050-122), 10× DPBS (purchased from Gibco, number Cat 14200-075), trypan blue dead cell dye (purchased from Lonza, number Cat 17-942E), trypsin-EDTA (10× Trypsin-EDTA, purchased from SIGMA, number Cat. 59427C, diluted 10-fold with 1× PBS). Bradford protein assay reagent. (purchased from Bio-Rad, number Cat,500-0006), and Pyruvate Colorimetric/Fluorometric Assay Kit (purchased from BioVision, number Cat K609)

6.2. Test Process

First, the mouse myoblasts C2C12 were inoculated in a 6-well culture plate containing 2 m/L of culture medium per well in a density of 1×10⁶ cells per well. The culture plate was placed in 5% CO₂ at 37° C. until 80% confluence. The culture medium was replaced with DMEM to induce the cells to differentiate into myotube cells.

The cells obtained after the differentiation were divided into three groups: a blank group, a control group, and an experimental group.

Experimental group: The plum extract sample 1 prepared by the method in the Example 1 was added into the cells obtained after the differentiation in a concentration of 0.03125 mg/mL, and then cultured at 37° C. for 48 h

Control group: The control sample A prepared by the method in the Example 1 was added into the cells obtained after the differentiation in a concentration of 0.03125 mg/mL, and then cultured at 37° C. for 48 h.

Blank group: without any treatment, that is, no additional compounds were added into the culture medium containing the fat cells obtained after the differentiation, and then cultured at 37° C. for 48 h.

Then, each group was washed with 1 mL of 1× PBS two times, cell lysis was 1.0 carried out with 100 μL/well Pyruvate Assay Buffer, centrifugation was carried out at 10000 g at 4° C. for 10 min, and then a supernatant was collected.

In addition, a standard curve was plotted for colorimetry: with pyruvic acid reference diluted to 1 nmol/μL, the standard curve was plotted for 0. 2, 4, 6, 8 and 10 nmol/well in a volume of 50 μL/well.

The to-be-tested sample in each group was added into 50 μL of reaction mixture (from the Pyruvate Colorimetric/Fluorometric Assay Kit) to react at room temperature for 30 min. After the reaction, the content of pyruvic acid was determined (by measuring an absorbance at 570 nm).

6.3, Experimental Results

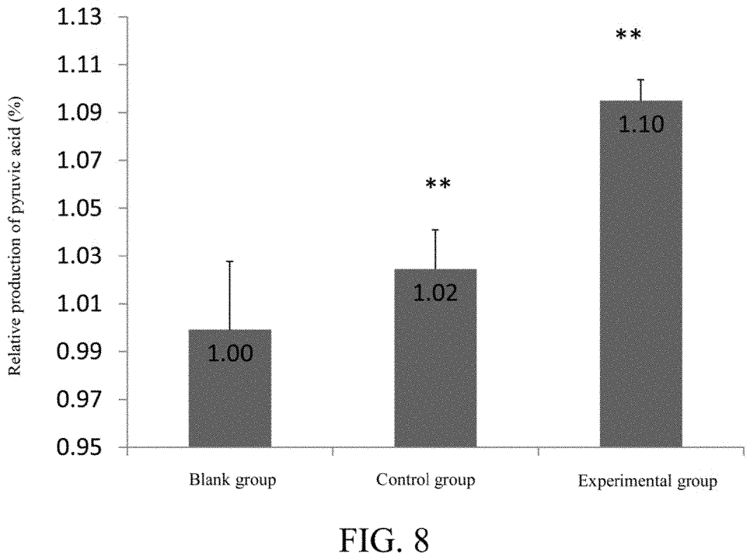

It can be learned from FIG. 8 that the production of pyruvic acid in the C2C12 cells in the experimental group is significantly increased by about 10%, indicating that the plum extract of the present invention can effectively increase the basal metabolic rate of cells, and the increase of the basal metabolic rate indicates the increase of the production rate of muscle cells and the reduction of accumulation of fat cells, thereby increasing the efficiency of glycolysis of cells.

Glycolysis is the metabolic pathway that converts glucose into pyruvic acid (CH₃COCOO—+ H+). The free energy released in this process is used to form the high-energy molecules adenosine triphosphate (ATP) and reduced nicotinamide adenine dinucleotide (NADH). The increase of the production of pyruvic acid in cells indicates the increase of the efficiency of glycolysis, allowing cells more efficient in generating energy and resulting in the increase of the basal metabolic rate. Therefore, the plum extract can effectively increase the efficiency of glycolysis, and helps prevent and relieve symptoms related to poor metabolism.

### Example 7: Human Subject Experiment

7.1. Sample: the plum extract sample 2 prepared in the Example 1.

7.2. Subject: eight subjects. These subjects had trouble with constipation or had a body fat percentage greater than 30%. That is, subjects with poor metabolism were selected for this test.

7.3. Test item: change of weight, change of waist circumference, and questionnaire on gastrointestinal conditions.

7.4. Test Method:

The eight subjects were given 2 g of plum extract sample 2 per day for two weeks. Measurement and questionnaire were separately carried out before drinking (week 0, also referred to as a control group) and after two weeks of drinking (week 2, also referred to as an experimental group).

Weights (kg) of these subjects were measured with a scale.

Waist circumferences (cm) of these subjects were measured with a tape measure.

A questionnaire on gastrointestinal conditions was filled out by these subjects at 2.0 week 0 and week 2. In the questionnaire, various gastrointestinal conditions were investigated, and the investigation and scores were shown in the following Table 2. Each subject was investigated for what happened before administration and during two weeks after administration of the plum extract to determine whether the following symptoms occurred. 1 represents complete disagreement, 2 represents disagreement, 3 represents partial disagreement, 4 represents fair, 5 represents partial agreement., 6 represents agreement, and 7 represents complete agreement.

TABLE 2

| | | | | | | | |
|---|---|---|---|---|---|---|---|
| Whether the symptoms occurred | 1 | 2 | 3 | 4 | 5 | 6 | 7 |

1. Overall gastrointestinal discomfort
2. Upper abdominal fullness
3. Nausea
4. Regurgitation
5. Frequency of intestinal peristalsis
6. Ease of defecation
7. Feces softening

In the following figures, the standard deviation is calculated by using the STDEV formula of Excel software, and whether there is a statistically significant difference is analyzed by one-tailed student t-test in Excel software. in the figures, “*” represents a p value less than 0.05, and “**” represents a p value less than 0.01. More “*” represents more significant statistical differences from the blank group.

7.5. Test Results

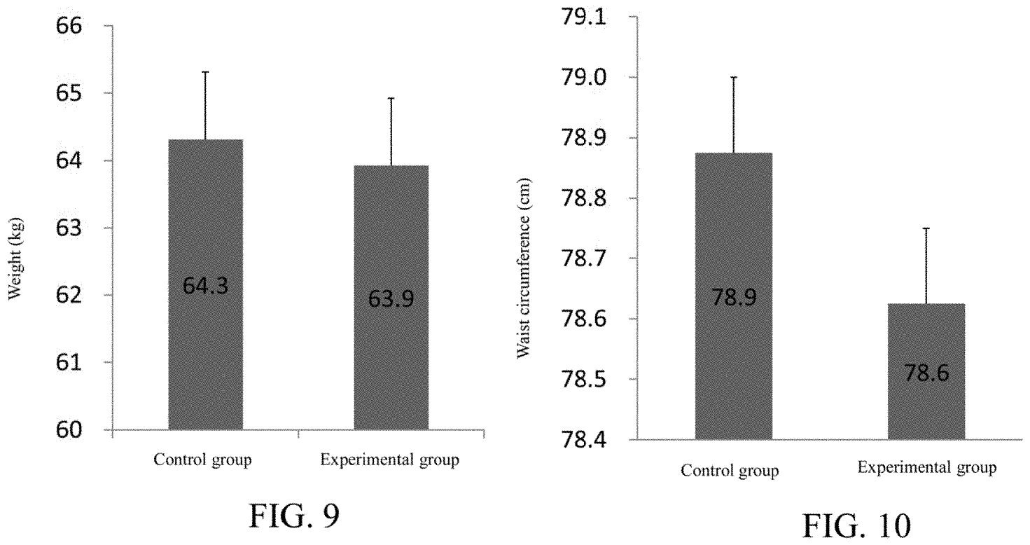

Referring to FIG. 9, after two weeks of taking 2 a of plum extract per day, the average weight of the eight subjects was decreased from 64.3 kg (at week 0) to 63.9 kg (at week 2) while maintaining daily diet and exercise. After taking the plum extract of the present invention for only two weeks, the average weight difference was 0.4 kg. That was, the intake of 2 g of plum extract per day effectively reduced weight and improve metabolism.

Referring to FIG. 10, after two weeks of taking 2 g of plum extract per day the average waist circumference of the eight subjects was decreased from 78.9 cm (at week 0) to 78.6 cm (at week 2) while maintaining daily diet and exercise. It can be learned that, after taking the plum extract of the present invention for only two weeks, the average waist circumference difference was 0.3 cm. That was, the intake of 2 g. of plum extract per day effectively reduced waist circumference, reduced abdominal fat accumulation, and improved metabolism.

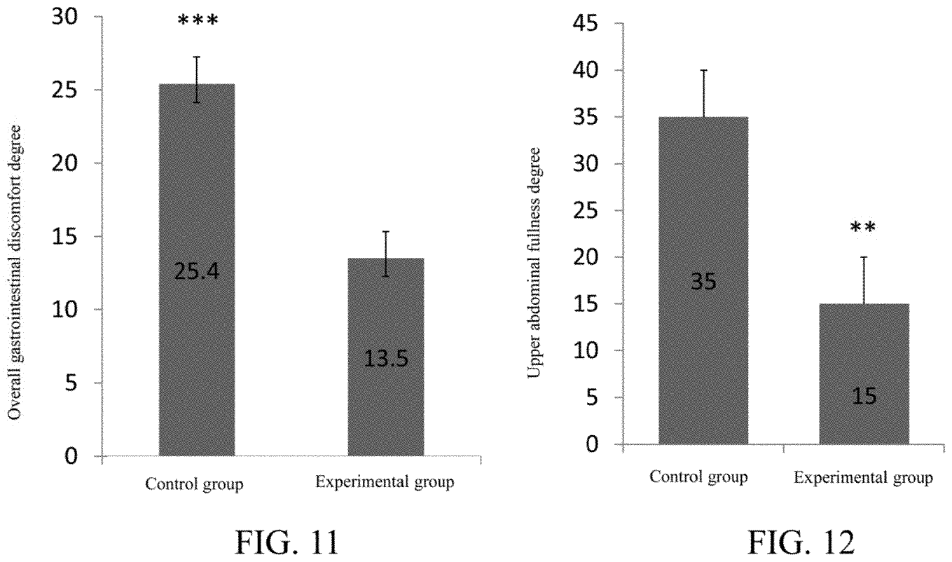

Referring to FIG. 11, for overall gastrointestinal discomfort, the average of the total scores of all the items for these subjects (that is, divided by 7) in Table 2 was decreased from 25.4 to 13.5, indicating that these subjects were self-assessed to have returned to no discomfort. The intake of 2 g of plum extract per day effectively relieved overall gastrointestinal discomfort by 46.9%.

Referring to FIG. 12, for upper abdominal fullness, the sum of the total scores for these subjects was decreased from 35 to 15.

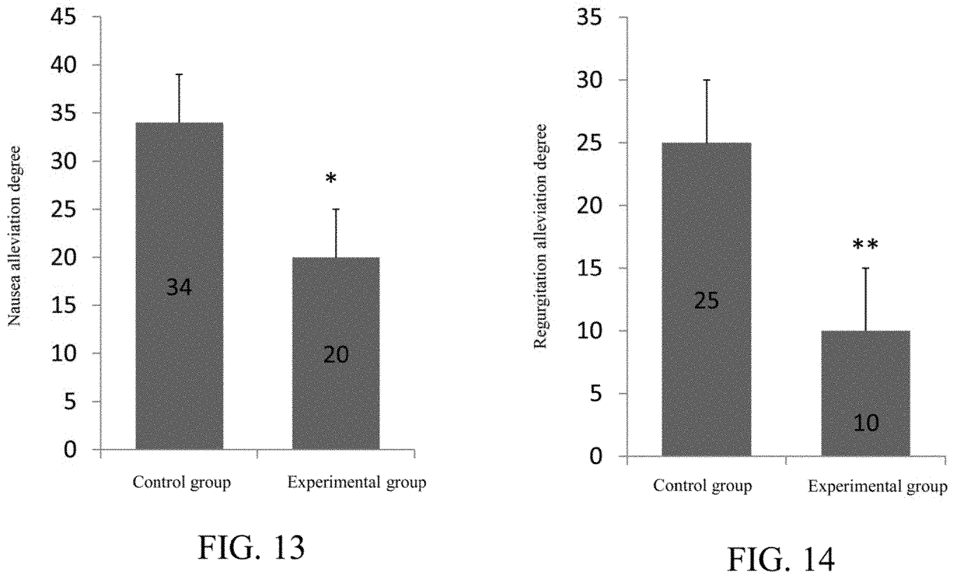

Referring to FIG. 13, for nausea, the sum of the total scores for these subjects was decreased from 34 to 20.

Referring to FIG. 14, for regurgitation, the sum of the total scores for these subjects was decreased from 25 to 10.

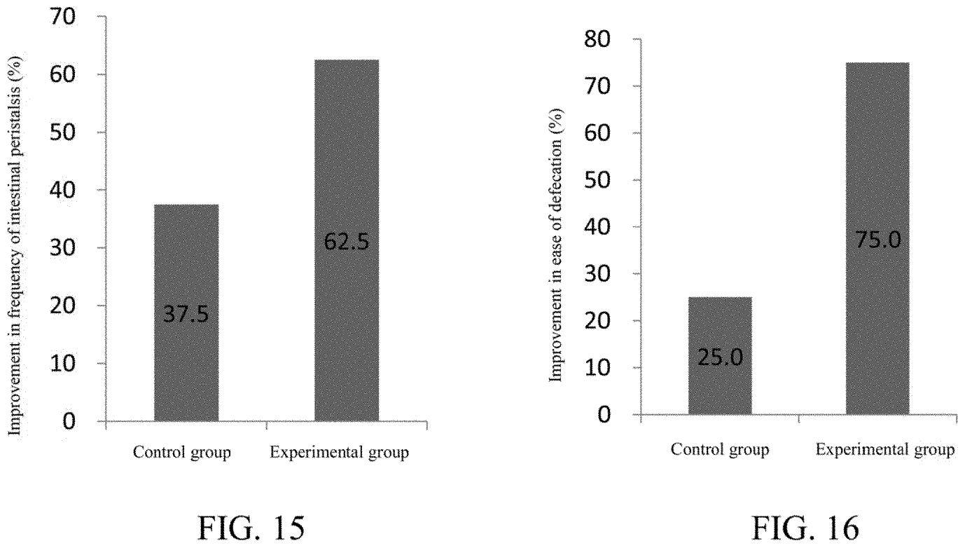

Referring to FIG. 15, for faster intestinal peristalsis, the sum of the total scores for these subjects was increased from 37.5 to 62.5.

Referring to FIG. 16, for ease of defecation, the sum of the total scores for these subjects was increased from 25 to 75.

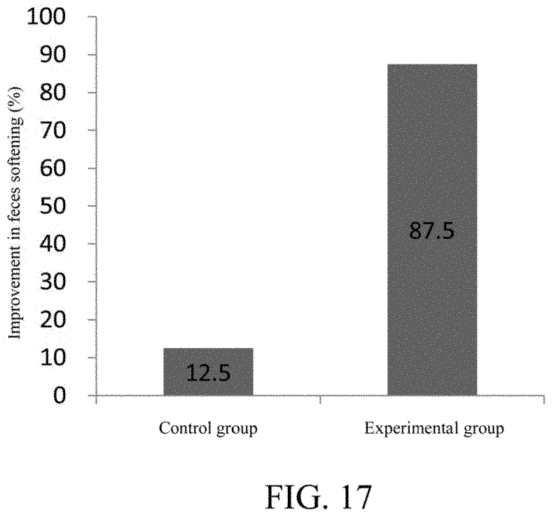

Referring to FIG. 17, for feces softening, the sum of the total scores for these subjects was increased from 12.5 to 87.5.

It can be learned above that the average score of each item was improved, indicating that, on average, the foregoing symptoms of these subjects are significantly relieved. The poor overall metabolism and the feeling of gastrointestinal discomfort were improved, and these subjects felt the improvement of their physical function.

It can be learned that the long-term use of plum extract can reduce weight, reduce waist circumference, relieve upper abdominal fullness, relieve nausea or regurgitation, increase the frequency of intestinal peristalsis, make ease of defecation, soften feces, and relieve overall gastrointestinal discomfort. That is, the plum extract has the effect of significantly improving body digestive function.

Based on the above, the plum extract according to any embodiment can improve physical function. The plum extract according to any embodiment can increase an expression level of a fat loss-related gene, that is, the LIPE gene, to improve physical function. The plum extract according to any embodiment can inhibit accumulation of lipid droplets in cells to improve physical function. The plum extract according to any embodiment can increase a metabolic rate by increasing an efficiency of pyruvic acid production to improve physical function. The plum extract according to any embodiment can reduce weight and/or waist circumference to improve physical function. The plum extract according to any embodiment can improve gastrointestinal function to improve physical function. In addition, the plum extract according to any embodiment can effectively improve physical function by taking 2 g of plum extract per day.

Although the present invention has been described in considerable detail with reference to certain preferred embodiments thereof, the disclosure is not for limiting the scope of the invention, Persons having ordinary skill in the art may make various modifications and changes without departing from the scope and spirit of the invention. Therefore, the scope of the appended claims should not be limited to the description of the preferred embodiments described above.

What is claimed is:

1. A method for improving physical function, comprising administering to a subject in need thereof a composition comprising a plum extract extracted from unripe green plums that are harvested within two to three months after flowering.

2. The method according to claim 1, wherein the improving physical function involves increasing an expression level of a fat loss-related gene in the subject, the fat loss-related gene comprises LIPE gene.

3. The method according to claim 2, wherein the improving physical function involves inhibiting accumulation of lipid droplets in cells of the subject.

4. The method according to claim 2, wherein the improving physical function involves increasing a metabolic rate of the subject.

5. The method according to claim 4, wherein the increasing a metabolic rate involves increasing an efficiency of pyruvic acid production of the subject.

6. The method according to claim 2, wherein the improving physical function involves reducing a weight of the subject and/or reducing a waist circumference of the subject.

7. The method according to claim 1, wherein the improving physical function involves improving gastrointestinal function of the subject.

8. The method according to claim 7, wherein the improving gastrointestinal function involves relieving abdominal distension, nausea, or regurgitation of the subject.

9. The method according to claim 7, wherein the improving gastrointestinal function involves improving, intestinal peristalsis, relieving constipation, or softening feces of the subject.

10. The method according to claim 6, wherein the composition is a food composition comprising at least 2 g/day of the plum extract.

11. The method according to claim 7, wherein the composition is a food composition comprising at least 2 g/day of the plum extract. 12, The method according to claim 8, wherein the composition is a food composition comprising at least 2 g/day of the plum extract.

13. The method according to claim 9, wherein composition is a food composition comprising at least 2 g/day of the plum extract.
